(19) United States
(12) Patent Application Publication
(10) Pub. No.: US 2023/0125467 A1
(43) Pub. Date: Apr. 27, 2023

(54) IMAGE-BASED HEALTH INDEX SCORING SYSTEM FOR GENITOURINARY TRACT

(71) Applicant: THE REGENTS OF THE UNIVERSITY OF CALIFORNIA, Oakland, CA (US)

(72) Inventors: Zhongping Chen, Irvine, CA (US); Yusi Miao, Irvine, CA (US); Yan Li, Irvine, CA (US); Bruce J. Tromberg, Irvine, CA (US); Yona Tadir, Irvine, CA (US)

(21) Appl. No.: 18/056,966

(22) Filed: Nov. 18, 2022

Related U.S. Application Data
(63) Continuation-in-part of application No. 16/235,426, filed on Dec. 28, 2018, now Pat. No. 11,517,194.
(60) Provisional application No. 62/611,990, filed on Dec. 29, 2017.

(51) Int. Cl. A61B 5/00; A61B 5/145; A61B 1/303; A61B 8/00; A61B 8/08; G06T 7/149; G06T 7/162; G06T 7/136

(52) U.S. Cl. CPC A61B 5/7275; A61B 5/0066; A61B 5/4337; A61B 5/14539; A61B 1/303; A61B 8/4416; A61B 8/485; G06T 7/149; G06T 7/162; G06T 7/136; G06T 2207/10101; G06T 2207/20081; G06T 2207/30004; G06T 2207/10132

(57) ABSTRACT

The present invention features a method for automated image-based prediction of physiologic and pathologic conditions of a vaginal wall of a patient using optical coherence tomography. In some embodiments, the method may comprise capturing, by a functional optical coherence tomography imaging probe, one or more images of the vaginal wall. The method may further comprise constructing a computing device a visualization of the vaginal wall, discretizing the visualization of the vaginal wall in to a discrete model, measuring a plurality of objective attributes from the discrete model, generating, based on the plurality of objective attributes, a Vaginal Health Index (VHI), generating an associated risk value based on the VHI and a plurality of patient attributes, reconstructing the visualization of the vaginal wall based on the associated risk value, and mapping the associated risk value to the visualization of the vaginal wall such that one or more at-risk areas are highlighted.

### CROSS-REFERENCES TO RELATED APPLICATIONS

This application is a continuation-in-part and claims benefit of U.S. Non-Provisional patent application Ser. No. 16/235,426 filed Dec. 28, 2018, which claims benefit of U.S. Provisional Patent Application No. 62/611,990 filed Dec. 29, 2017, the specifications of which are incorporated herein in their entirety by reference.

### GOVERNMENT SUPPORT

This invention was made with government support under Grant No. P41EB-015890, FG18869 (443810/29873), awarded by NIH. The government has certain rights in the invention.

### FIELD OF THE INVENTION

The present invention is directed to the vaginal health index (VHI) scoring system. More specifically, it relates to a methodology for an automated image-based prediction of physiologic and pathologic conditions of the vaginal wall using functional optical coherence tomography (F-OCT), including but not limited to OCT, Doppler OCT, polarization sensitive OCT (PS-OCT), and optical coherence elastography (OCE). Some of them are currently defined by subjective means such as the VHI score. The VHI score index system can be used for screen, therapy guidance and optimization, and therapy evaluation.

### BACKGROUND OF THE INVENTION

Genitourinary syndrome of menopause (GSM) is a broad medical term for various conditions such as vulvovaginal atrophy, urinary disorders, burning, itching, and other symptoms. GSM is among the most common clinical complaints of women, affecting nearly half of post-menopausal women and negatively impacting their quality-of-life, as a result of a physiological decline in estrogen. However, GSM is not widely recognized as a disease and is often left undiagnosed until the later stage, compared to other pathologic conditions of female reproductive organs such as cervical intraepithelial neoplasia (CIN), cervical cancer, and other well defined, and accurately staged conditions.

The current evaluation of vaginal health for patients with GSM lacks objective tissue assessment to address tissue morphology, blood vessel formation, and elasticity related to tissue integrity. At present, GSM diagnosis is based on the patient's medical history and physical exam. Clinicians may use a scoring system known as the Vaginal Health Index (VHI) that evaluates vaginal health based on vaginal elasticity, fluid volume, pH, epithelial integrity, and moisture on a scale of 1-5; however, it's vulnerable to inter- and intra-observer variation. Other laboratory tests exist, such as vaginal cytology (Maturation Index) and wet mount, but neither provide direct, comprehensive information about tissue quality and severity status. Furthermore, these scores may not correlate with subjective discomfort on a Visual Analog Scale (VAS) or validated questionnaires. Questionnaires that have demonstrated utility in assessing the severity of GSM symptoms include Vulvovaginal Symptom Questionnaire (VSQ), Female Sexual Function Index (FSFI), Pelvic Organ Prolapse/Urinary Incontinence Sexual Questionnaire (PISQ), and Pelvic Floor Disorders Inventory (PFDI). Beyond the architectural components, we know that the vaginal microbiome changes in menopause are restored with vaginal estrogen: however, data are lacking to determine whether other treatment modalities such as various hormonal supplementation, non-medicated moisturizers, or energy-based devices can achieve similar results. Without clinical justification to perform an invasive biopsy for GSM, current published data lack robust, objective information regarding the anatomical changes associated with menopause and its treatments. This is where non-invasive F-OCT technology would provide significant value as an objective, and likely reliable, outcome measure that may be used as part of a composite score to determine vaginal health, assist in patient selection for various treatment modalities, guide the treatment, and predict and evaluate the efficacy of treatment.

### BRIEF SUMMARY OF THE INVENTION

It is an objective of the present invention to provide methods that allow for automated image-based prediction of physiologic and pathologic conditions of a vaginal wall of a patient using F-OCT, as specified in the independent claims. Embodiments of the invention are given in the dependent claims. Embodiments of the present invention can be freely combined with each other if they are not mutually exclusive.

The present invention features a method for automated image-based prediction of physiologic and pathologic conditions of a vaginal wall of a patient using optical coherence tomography. In some embodiments, the method may comprise capturing, by an F-OCT imaging probe, one or more images of the vaginal wall. The method may further comprise constructing, by a computing device, a visualization of the vaginal wall based on a segmentation algorithm. The computing device may comprise a processor capable of executing computer-readable instructions, and a memory component comprising a plurality of computer-readable instructions capable of being executed by the processor. The method may further comprise discretizing, by the computing device, the visualization of the vaginal wall into a discrete model. The method may further computing device that determine microvascular density of vaginal wall. The method may further computing device that determine tissue birefringence of vaginal wall. The method may further computing device that determine elasticity mapping of vaginal wall. The method may further comprise the computing device measuring a plurality of objective attributes from the discrete model, generating, based on the plurality of objective attributes, a Vaginal Health Index (VHI), generating an associated risk value based on the VHI and a plurality of patient attributes, reconstructing the visualization of the vaginal wall based on the associated risk value, and mapping the associated risk value to the visualization of the vaginal wall such that one or more at-risk areas are highlighted.

One of the unique and inventive technical features of the present invention is the automatic generation of a vaginal health index score based on a plurality of F-OCT probe images. Without wishing to limit the invention to any theory or mechanism, it is believed that the technical feature of the present invention advantageously provides for time- and resource-efficient determination of a patient's vaginal health without any need for human calculation or intervention. None of the presently known prior references or work has the unique inventive technical feature of the present invention.

Prior systems generally teach the observation of a plurality of subjective parameters whereas the present invention assesses VHI based on quantitative image-based measurements.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a flow chart of the method for automated image-based prediction of physiologic and pathologic conditions of a vaginal wall of a patient using F-OCT of the present invention.

FIG. 1B shows a schematic of a system for carrying out the method of the present invention.

FIG. 2 shows a table of some aspects of calculating a vaginal health index.

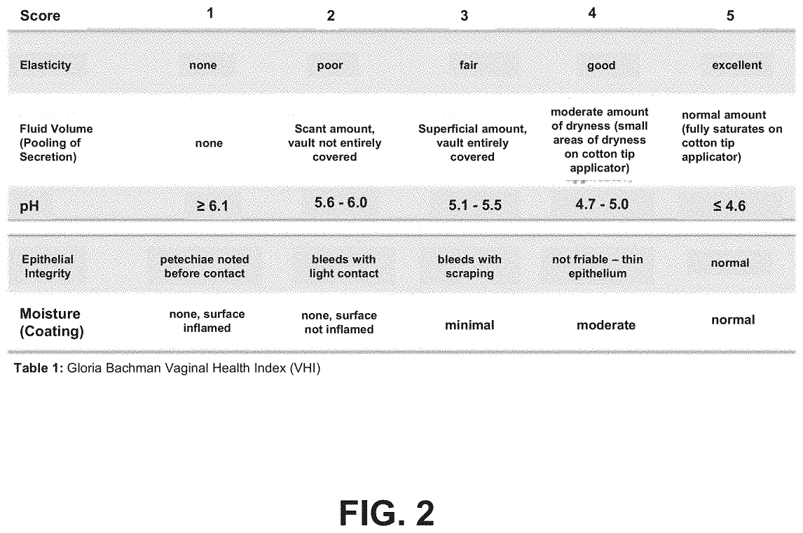

FIG. 3 shows a table of some aspects of a vaginal health index mapped to the parameters required to score the said aspects.

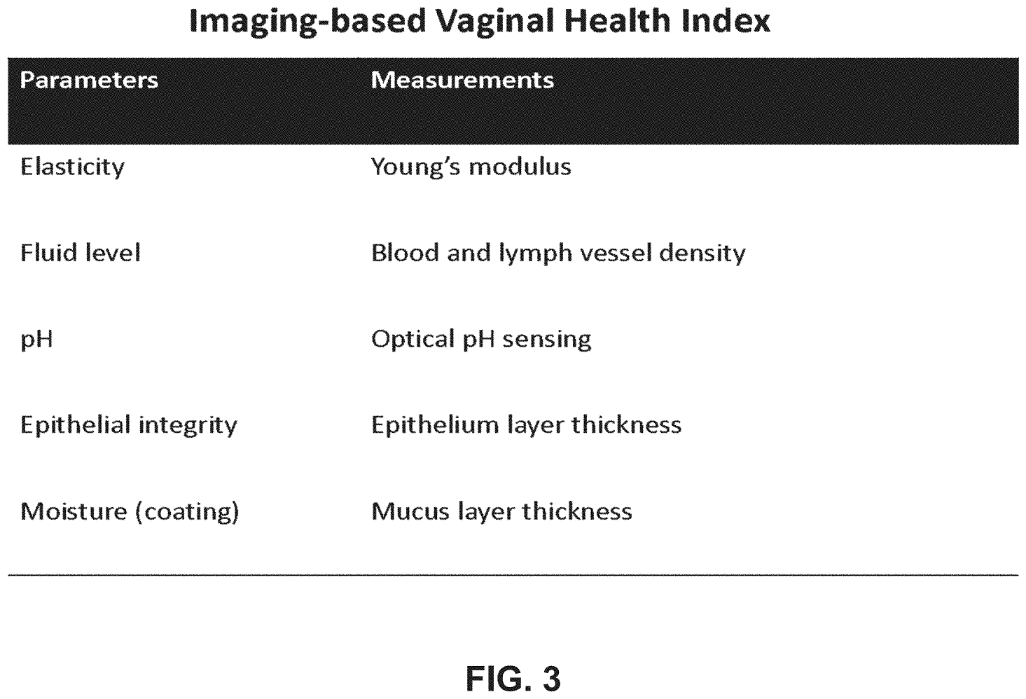

### DETAILED DESCRIPTION OF THE INVENTION

Following is a list of elements corresponding to a particular element referred to herein:

100 F-OCT probe

200 computing device

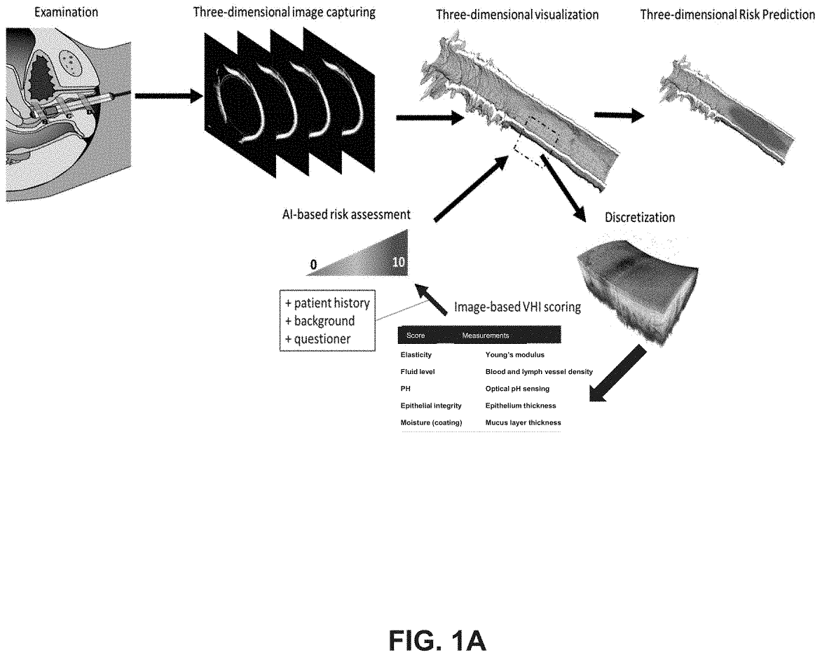

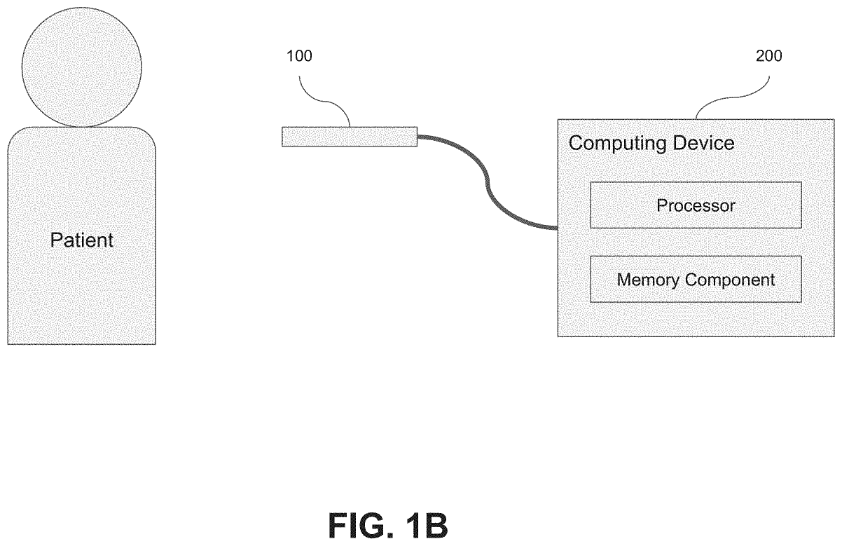

Referring now to FIG. 1A, the present invention features a method for automated image-based prediction of physiologic and pathologic conditions of a vaginal wall of a patient using F-OCT. In some embodiments, the method may comprise capturing, by a F-OCT imaging probe (100), one or more images of the vaginal wall. The method may further comprise constructing, by a computing device (200), a visualization of the vaginal wall based on a segmentation algorithm. The computing device (200) may comprise a processor capable of executing computer-readable instructions, and a memory component comprising a plurality of computer-readable instructions capable of being executed by the processor. The method may further comprise discretizing, by the computing device (200), the visualization of the vaginal wall into a discrete model. The method may further computing device that determine microvascular density of vaginal wall. The method may further computing device that determine tissue birefringence of vaginal wall. The method may further computing device that determine elasticity mapping of vaginal wall. The method may further comprise the computing device (200) measuring a plurality of objective attributes from the discrete model, generating, based on the plurality of objective attributes, a Vaginal Health Index (VHI), generating an associated risk value based on the VHI and a plurality of patient attributes, reconstructing the visualization of the vaginal wall based on the associated risk value, and mapping the associated risk value to the visualization of the vaginal wall such that one or more at-risk areas are highlighted.

In some embodiments, the patient attributes may comprise age, microvascular density of vaginal wall, tissue birefringence of vaginal wall, vaginal elasticity, vaginal secretions, pH, epithelial mucous membrane, epithelial thickness, vaginal hydration, or a combination thereof. In some embodiments, the F-OCT imaging probe (100) may comprise a three-dimensional F-OCT scanner. In other embodiments, the F-OCT imaging probe (100) may comprise a two-dimensional F-OCT scanner, an F-OCT point scanner, or a combination thereof. In some embodiments, the visualization of the vaginal wall may comprise a three-dimensional visualization, and the segmentation algorithm may comprise machine learning segmentation, graph-based segmentation, thresholding, image filters, active counter-image segmentation, or a combination thereof. In F-OCT cross-sectional image, the area corresponding to a specific tissue (such as epithelium) is automatically detected and highlighted by a segmentation algorithm. Then, the three-dimensional visualization of the segmented area is created by interpolating the segmentation of all the F-OCT images. In some embodiments, generating the associated risk value may comprise implementing a convolutional neural network, a linear regression model, a logistic regression model, a random forest algorithm, a thresholding algorithm, or a combination thereof.

In some embodiments, the method may further comprise measuring, by an optical coherence elastography (OCE) imaging probe, a Young's Modulus measurement, and the plurality of objective attributes may comprise the Young's Modulus measurement. Measuring the Young's Modulus, by the OCE imaging probe, may comprise an ultrasound-based OCE technique, a piezo-based OCE technique, an air-puff OCE technique, a laser-excitation OCE technique, a magneto-motive OCE technique, or a combination thereof. Information is gathered by a patient based on F-OCT measurement. In OCE, a mechanical force is applied to a specific tissue location, and the miron-scale tissue deformation is observed using F-OCT.

In some embodiments, the method may further comprise measuring a vaginal fluid level value and the plurality of objective attributes may comprise the vaginal fluid level value and microvascular density. In some embodiments, measuring the vaginal fluid level value and microvascular density may comprise optical coherence angiography, Doppler OCT, or a lymphatic vessel visualization technique.

In some embodiments, the method may further comprise measuring, based on an optical sensing technique, an electrical sensing technique, or a combination thereof, a pH measurement and the plurality of objective attributes may comprise the pH measurement. The optical sensing technique may comprise F-OCT, including OCT, PS-OCT, Doppler OCT, OCT angiography, or OCE. In some embodiments, the method may further comprise measuring, by the F-OCT imaging probe (100), a tissue thickness measurement, and the plurality of objective attributes may comprise the tissue thickness measurement. In some embodiments, the method may further comprise measuring, by the F-OCT imaging probe (100), an epithelium thickness measurement and the plurality of objective attributes may comprise the epithelium thickness measurement. In some embodiments, the method may further comprise measuring, by the F-OCT imaging probe (100), a lamina propria thickness measurement and the plurality of objective attributes may comprise the lamina propria thickness measurement.

In some embodiments, the method may further comprise measuring, by a PS-OCT probe, an extracellular matrix (ECM) content value and the plurality of objective attributes may comprise the extracellular matrix (ECM) content value. In some embodiments, the method may further comprise measuring, by a texture analysis technique, a cellular hydration measurement and the plurality of objective attributes may comprise the cellular hydration measurement. In some embodiments, the method may further comprise measuring an attenuation coefficient measurement and the plurality of objective attributes may comprise the attenuation coefficient measurement. The attenuation coefficient measurement shows how quickly incident light is attenuated when passing through tissue. It enables quantitative analysis of tissue properties from OCT signals. In some embodiments, the method may further comprise measuring, by the F-OCT imaging probe (100), a quantification of vaginal folding and the plurality of objective attributes may comprise the quantification of vaginal folding. In some embodiments, the method may further comprise measuring, by the F-OCT imaging probe (100), a rete pegs density and the plurality of objective attributes may comprise the rete pegs density.

Referring now to FIG. 1B, the present invention features a system for automated image-based prediction of physiologic and pathologic conditions of a vaginal wall of a patient using F-OCT system. In some embodiments, the system may comprise an F-OCT imaging probe, and a computing device (200) communicatively coupled to the F-OCT imaging probe. In some embodiments, the computing device (200) may comprise a processor capable of executing computer-readable instructions, and a memory component comprising computer-readable instructions. In some embodiments, the computer-readable instructions may comprise capturing, by the F-OCT imaging probe (100), one or more images of the vaginal wall, constructing a visualization of the vaginal wall based on a segmentation algorithm, discretizing the visualization of the vaginal wall in to a discrete model, measuring a plurality of objective attributes from the discrete model, generating based on the plurality of objective attributes, a VHI, generating an associated risk value based on the VHI and a plurality of patient attributes, reconstructing the visualization of the vaginal wall based on the associated risk value, and mapping the associated risk value to the visualization of the vaginal wall such that one or more at-risk areas are highlighted.

In some embodiments, the F-OCT imaging probe (100) may comprise a three-dimensional F-OCT scanner. In other embodiments, the OCT imaging probe (100) may comprise a two-dimensional F-OCT scanner, an F-OCT point scanner, or a combination thereof. In some embodiments, the visualization of the vaginal wall may comprise a three-dimensional visualization, and the segmentation algorithm may comprise machine learning segmentation, graph-based segmentation, thresholding, image filters, active counter image segmentation, or a combination thereof.

In some embodiments, the system may further comprise an optical coherence elastography (OCE) imaging probe, and the memory component may further comprise instructions for measuring, by the OCE imaging probe, a Young's Modulus measurement, and the plurality of objective attributes may comprise the Young's Modulus measurement. Measuring the Young's Modulus measurement, by the OCE imaging probe, may comprise an ultrasound-based OCE technique, a piezo-based OCE technique, an air-puff OCE technique, a laser-excitation OCE technique, a magneto-motive OCE technique, or a combination thereof. In some embodiments, generating the associated risk value comprises implementing a convolutional neural network, a linear regression model, a logistic regression model, a random forest algorithm, a thresholding algorithm, or a combination thereof.

In some embodiments, the computer-readable instructions may further comprise measuring a vaginal fluid level value and the plurality of objective attributes may comprise the vaginal fluid level value. In some embodiments, measuring the vaginal fluid level value may comprise optical coherence angiography (OCTA), Doppler OCT, or a lymphatic vessel visualization technique.

In some embodiments, the computer-readable instructions may further comprise measuring, based on an optical sensing technique, an electrical sensing technique, or a combination thereof, a pH measurement and the plurality of objective attributes may comprise the pH measurement. In some embodiments, the computer-readable instructions may further comprise measuring, by the F-OCT imaging probe (100), a tissue thickness measurement, and the plurality of objective attributes may comprise the tissue thickness measurement. In some embodiments, the computer-readable instructions may further comprise measuring, by the F-OCT imaging probe (100), an epithelium thickness measurement and the plurality of objective attributes may comprise the epithelium thickness measurement. In some embodiments, the computer-readable instructions may further comprise measuring, by the F-OCT imaging probe (100), a lamina propria thickness measurement and the plurality of objective attributes may comprise the lamina propria thickness measurement.

In some embodiments, the present invention may comprise using the associated risk value and the corresponding image to screen patients who will most likely benefit from the energy based treatment, including but not limited to laser treatment. In some embodiments, the present invention may comprise using the associated risk value and corresponding image to plan, guide, and optimize the energy based treatment, including but not limited to laser treatment. In some embodiments, the present invention may comprise using the associated risk value and corresponding imaging to evaluate efficacy of energy based treatment, including but not limited to laser treatment. In some embodiments, implementing the associated risk value and the corresponding image to screen patients who will most likely benefit from energy-based treatments including but not limited to laser treatment comprises the use of a convolutional neural network, a linear regression model, a logistic regression model, a random forest algorithm, a thresholding algorithm, or a combination thereof. In some embodiments, implementing the associated risk value and corresponding image to plan, guide, and optimize energy-based treatments including but not limited to laser treatment comprises use of a convolutional neural network, a linear regression model, a logistic regression model, a random forest algorithm, a thresholding algorithm, or a combination thereof. In some embodiments, implementing the associated risk value and corresponding imaging to evaluate efficacy of energy-based treatments including but not limited to laser treatment comprises the use of a convolutional neural network, a linear regression model, a logistic regression model, a random forest algorithm, a thresholding algorithm, or a combination thereof.

In some embodiments, the computer-readable instructions may further comprise measuring, by a polarization-sensitive OCT probe, an extracellular matrix (ECM) content value and the plurality of objective attributes may comprise the extracellular matrix (ECM) content value. In some embodiments, the computer-readable instructions may further comprise measuring, by a texture analysis technique, a cellular hydration measurement and the plurality of objective attributes may comprise the cellular hydration measurement. In some embodiments, the computer-readable instructions may further comprise measuring an attenuation coefficient measurement and the plurality of objective attributes may comprise the attenuation coefficient measurement. In some embodiments, the computer-readable instructions may further comprise measuring, by the F-OCT imaging probe (100), a quantification of vaginal folding and the plurality of objective attributes may comprise the quantification of vaginal folding. In some embodiments, the computer-readable instructions may further comprise measuring, by the F-OCT imaging probe (100), a rete pegs density and the plurality of objective attributes may comprise the rete pegs density.

The invention describes a methodology and workflow for image-based objective vaginal health index (VHI) scoring system based on F-OCT imaging and mapping of VHI to the three-dimensional tissue segmentation. The illustrated embodiment of the invention includes a non-invasive examination of the vaginal canal using an F-OCT imaging probe, capturing of three-dimensional F-OCT data, three-dimensional tissue segmentation, computer-aided prediction of VHI sore and associated risk, and mapping of predicted risk to a three-dimensional structure. The traditional VHI scoring system based primarily on a clinician's judgment is replaced with objective image-based measurement results.

In another embodiment, F-OCT information can also be acquired using a two-dimensional scanner or a point-scanning. In some embodiments, the F-OCT imaging probe is communicatively coupled to the computing device by a wired connection. In other embodiments, the OCT imaging probe is communicatively coupled to the computing device by a wireless connection comprising WiFi, Bluetooth, ZigBee, NFC, WiMAX, LTE, HSPA, EV-DO, or any other form of wireless communication. The F-OCT imaging probe and computing device may comprise compatible communication components to allow for the aforementioned communicative coupling methods.

The computer system can include a desktop computer, a laptop computer, a tablet, or the like and can include digital electronic circuitry, firmware, hardware, memory, a computer storage medium, a computer program, a processor (including a programmed processor), or the like. The computing system may include a desktop computer with a screen and a tower. The tower can store digital images in binary form. The data/images can also be stored in the cloud. The images can also be divided into a matrix of pixels. The pixels can include a digital value of one or more bits, defined by the bit depth. The network or a direct connection interconnects the imaging apparatus and the computer system.

The term “processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user. Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation, a SPARC processor made by Sun Microsystems, an Athlon, Sempron, Phenom, or Opteron processor made by AMD Corporation, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation; the Mac OS X operating system from Apple Computer Corp.; a Unix or Linux-type operating system available from many vendors or what is referred to as an open source; another or a future operating system; or some combination thereof. An operating system interfaces with firmware and hardware in a well-known manner and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

The following embodiments are intended to be illustrative only and not to be limiting in any way.

### Embodiment 1

A method for automated image-based prediction of physiologic and pathologic conditions of a vaginal wall of a patient using functional optical coherence tomography (F-OCT), the method comprising:
a. capturing, by an functional optical coherence tomography (F-OCT) imaging probe (100), one or more images of the vaginal wall;
b. constructing, by a computing device (200), a visualization of the vaginal wall based on a segmentation algorithm;
c. discretizing, by the computing device (200), the visualization of the vaginal wall in to a discrete model;
d. measuring, by the computing device (200), a plurality of objective attributes from the discrete model;
e. generating, by the computing device (200), based on the plurality of objective attributes, a Vaginal Health Index (VHI);
f. generating, by the computing device (200), an associated risk value based on the VHI and a plurality of patient attributes;
g. reconstructing, by the computing device (200), the visualization of the vaginal wall based on the associated risk value; and
h. mapping the associated risk value to the visualization of the vaginal wall such that one or more at-risk areas are highlighted.

### Embodiment 2

The method of embodiment 1, wherein the OCT imaging probe (100) comprises a three-dimensional OCT scanner.

### Embodiment 3

The method of embodiment 1, wherein the OCT imaging probe (100) comprises a two-dimensional OCT scanner, an OCT point scanner, or a combination thereof.

### Embodiment 4

The method of embodiment 2, wherein the visualization of the vaginal wall comprises a three-dimensional visualization, wherein the segmentation algorithm comprises machine learning segmentation, graph-based segmentation, thresholding, image filters, active counter image segmentation, or a combination thereof.

### Embodiment 5

The method of embodiment 1 further comprising:
a. measuring, by an optical coherence elastography (OCE) imaging probe, a Young's Modulus measurement;
   wherein the plurality of objective attributes comprises the Young's Modulus measurement.

### Embodiment 6

The method of embodiment 5, wherein measuring the Young's Modulus measurement, by the OCE imaging probe, comprises an ultrasound-based OCE technique, a piezo-based OCE technique, an air-puff OCE technique, a laser-excitation OCE technique, a magneto-motive OCE technique, or a combination thereof.

### Embodiment 7

The method of embodiment 1 further comprising:
a. measuring a vaginal fluid level value;
   wherein the plurality of objective attributes comprises the vaginal fluid level value.

### Embodiment 8

The method of embodiment 7, wherein measuring the vaginal fluid level value comprises optical coherence angiography, Doppler OCT, or a lymphatic vessel visualization technique.

### Embodiment 9

The method of embodiment 1 further comprising:
a. measuring, based on an optical sensing technique, an electrical sensing technique, or a combination thereof, a pH measurement;
   wherein the plurality of objective attributes comprises the pH measurement.

### Embodiment 10

The method of embodiment 7, further comprising further comprise measuring, by a polarization-sensitive OCT probe, an extracellular matrix (ECM) content value;
wherein the plurality of objective attributes may comprise the extracellular matrix (ECM) content value.

### Embodiment 11

The method of embodiment 1, wherein generating the associated risk value comprises implementing a convolutional neural network, a linear regression model, a logistic regression model, a random forest algorithm, a thresholding algorithm, or a combination thereof.

### Embodiment 12

A system for automated image-based prediction of physiologic and pathologic conditions of a vaginal wall of a patient using F-OCT, the system comprising:
a. an F-OCT imaging probe; and
b. a computing device (200) communicatively coupled to the OCT imaging probe, comprising a processor capable of executing computer-readable instructions, and a memory component comprising computer-readable instructions for:
   i. capturing, by the F-OCT imaging probe (100), one or more images of the vaginal wall;
   ii. constructing a visualization of the vaginal wall based on a segmentation algorithm;
   iii. discretizing the visualization of the vaginal wall in to a discrete model;
   iv. measuring a plurality of objective attributes from the discrete model;
   v. generating based on the plurality of objective attributes, a Vaginal Health Index (VHI);
   vi. generating an associated risk value based on the VHI and a plurality of patient attributes;
   vii. reconstructing the visualization of the vaginal wall based on the associated risk value; and
   viii. mapping the associated risk value to the visualization of the vaginal wall such that one or more at-risk areas are highlighted.

### Embodiment 13

The system of embodiment 12, wherein the F-OCT imaging probe (100) comprises a three-dimensional F-OCT scanner.

### Embodiment 14

The system of embodiment 12, wherein the F-OCT imaging probe (100) comprises a two-dimensional F-OCT scanner, an F-OCT point scanner, or a combination thereof.

### Embodiment 15

The system of embodiment 13, wherein the visualization of the vaginal wall comprises a three-dimensional visualization, wherein the segmentation algorithm comprises machine learning segmentation, graph-based segmentation, thresholding, image filters, active counter image segmentation, or a combination thereof.

### Embodiment 16

The system of embodiment 12 further comprising an optical coherence elastography (OCE) imaging probe, wherein the memory component further comprises instructions for measuring, by the optical coherence elastography (OCE) imaging probe, a Young's Modulus measurement, wherein the plurality of objective attributes comprises the Young's Modulus measurement.

### Embodiment 17

The system of embodiment 16, wherein measuring the Young's Modulus measurement, by the OCE imaging probe, comprises an ultrasound-based OCE technique, a piezo-based OCE technique, an air-puff OCE technique, a laser-excitation OCE technique, a magneto-motive OCE technique, or a combination thereof.

### Embodiment 18

The system of embodiment 12, wherein the memory component further comprises instructions for:
a. measuring a vaginal fluid level value;
   wherein the plurality of objective attributes comprises the vaginal fluid level value.

### Embodiment 19

The system of embodiment 18, wherein measuring the vaginal fluid level value comprises optical coherence angiography, Doppler OCT, or a lymphatic vessel visualization technique.

### Embodiment 20

The system of embodiment 12, wherein the memory component further comprises instructions for:
a. measuring, based on an optical sensing technique, an electrical sensing technique, or a combination thereof, a pH measurement;
   wherein the plurality of objective attributes comprises the pH measurement.

### Embodiment 21

The system of embodiment 12, further comprising further comprise measuring, by a polarization-sensitive OCT probe, an extracellular matrix (ECM) content value;
wherein the plurality of objective attributes may comprise the extracellular matrix (ECM) content value.

### Embodiment 22

The system of embodiment 12, wherein generating the associated risk value comprises implementing a convolutional neural network, a linear regression model, a logistic regression model, a random forest algorithm, a thresholding algorithm, or a combination thereof.

### Embodiment 23

The system of embodiment 12, further comprising implementing the associated risk value and the corresponding image to screen patients who will most likely benefit from laser treatment through use of a convolutional neural network, a linear regression model, a logistic regression model, a random forest algorithm, a thresholding algorithm, or a combination thereof.

### Embodiment 24

The system of embodiment 12, further comprising implementing the associated risk value and corresponding image to plan, guide, and optimize laser treatment through use of a convolutional neural network, a linear regression model, a logistic regression model, a random forest algorithm, a thresholding algorithm, or a combination thereof.

### Embodiment 25

The system of embodiment 12, further comprising implementing the associated risk value and corresponding imaging to evaluate efficacy of laser treatment through use of a convolutional neural network, a linear regression model, a logistic regression model, a random forest algorithm, a thresholding algorithm, or a combination thereof.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for automated image-based prediction of physiologic and pathologic conditions of a vaginal wall of a patient using functional optical coherence tomography (F-OCT), the method comprising:
a. capturing, by an functional optical coherence tomography (F-OCT) imaging probe (100), one or more images of the vaginal wall;
b. constructing, by a computing device (200), a visualization of the vaginal wall based on a segmentation algorithm;
c. discretizing, by the computing device (200), the visualization of the vaginal wall in to a discrete model;
d. measuring, by the computing device (200), a plurality of objective attributes from the discrete model;
e. generating, by the computing device (200), based on the plurality of objective attributes, a Vaginal Health Index (VHI);
f. generating, by the computing device (200), an associated risk value based on the VHI and a plurality of patient attributes;
g. reconstructing, by the computing device (200), the visualization of the vaginal wall based on the associated risk value; and
h. mapping the associated risk value to the visualization of the vaginal wall such that one or more at-risk areas are highlighted.

2. The method of claim 1, wherein the OCT imaging probe (100) comprises a three-dimensional OCT scanner.

3. The method of claim 1, wherein the OCT imaging probe (100) comprises a two-dimensional OCT scanner, an OCT point scanner, or a combination thereof.

4. The method of claim 2, wherein the visualization of the vaginal wall comprises a three-dimensional visualization, wherein the segmentation algorithm comprises machine learning segmentation, graph-based segmentation, thresholding, image filters, active counter image segmentation, or a combination thereof.

5. The method of claim 1 further comprising:
a. measuring, by an optical coherence elastography (OCE) imaging probe, a Young's Modulus measurement;
wherein the plurality of objective attributes comprises the Young's Modulus measurement.

6. The method of claim 5, wherein measuring the Young's Modulus measurement, by the OCE imaging probe, comprises an ultrasound-based OCE technique, a piezo-based OCE technique, an air-puff OCE technique, a laser-excitation OCE technique, a magneto-motive OCE technique, or a combination thereof.

7. The method of claim 1 further comprising:
a. measuring a vaginal fluid level value;
wherein the plurality of objective attributes comprises the vaginal fluid level value.

8. The method of claim 7, wherein measuring the vaginal fluid level value comprises optical coherence angiography, Doppler OCT, or a lymphatic vessel visualization technique.

9. The method of claim 1 further comprising:
a. measuring, based on an optical sensing technique, an electrical sensing technique, or a combination thereof, a pH measurement;
wherein the plurality of objective attributes comprises the pH measurement.

10. The method of claim 7, further comprising further comprise measuring, by a polarization-sensitive OCT probe, an extracellular matrix (ECM) content value;
wherein the plurality of objective attributes may comprise the extracellular matrix (ECM) content value.

11. The method of claim 1, wherein generating the associated risk value comprises implementing a convolutional neural network, a linear regression model, a logistic regression model, a random forest algorithm, a thresholding algorithm, or a combination thereof.

12. A system for automated image-based prediction of physiologic and pathologic conditions of a vaginal wall of a patient using F-OCT, the system comprising:
a. an F-OCT imaging probe; and
b. a computing device (200) communicatively coupled to the OCT imaging probe, comprising a processor capable of executing computer-readable instructions, and a memory component comprising computer-readable instructions for:
i. capturing, by the F-OCT imaging probe (100), one or more images of the vaginal wall;
ii. constructing a visualization of the vaginal wall based on a segmentation algorithm;
iii. discretizing the visualization of the vaginal wall in to a discrete model;
iv. measuring a plurality of objective attributes from the discrete model;
v. generating based on the plurality of objective attributes, a Vaginal Health Index (VHI);
vi. generating an associated risk value based on the VHI and a plurality of patient attributes;
vii. reconstructing the visualization of the vaginal wall based on the associated risk value; and
viii. mapping the associated risk value to the visualization of the vaginal wall such that one or more at-risk areas are highlighted.

13. The system of claim 12, wherein the F-OCT imaging probe (100) comprises a three-dimensional F-OCT scanner.

14. The system of claim 12, wherein the F-OCT imaging probe (100) comprises a two-dimensional F-OCT scanner, an F-OCT point scanner, or a combination thereof.

15. The system of claim 13, wherein the visualization of the vaginal wall comprises a three-dimensional visualization, wherein the segmentation algorithm comprises machine learning segmentation, graph-based segmentation, thresholding, image filters, active counter image segmentation, or a combination thereof.

16. The system of claim 12 further comprising an optical coherence elastography (OCE) imaging probe, wherein the memory component further comprises instructions for measuring, by the optical coherence elastography (OCE) imaging probe, a Young's Modulus measurement, wherein the plurality of objective attributes comprises the Young's Modulus measurement.

17. The system of claim 16, wherein measuring the Young's Modulus measurement, by the OCE imaging probe, comprises an ultrasound-based OCE technique, a piezo-based OCE technique, an air-puff OCE technique, a laser-excitation OCE technique, a magneto-motive OCE technique, or a combination thereof.

18. The system of claim 12, wherein the memory component further comprises instructions for:
a. measuring a vaginal fluid level value;
wherein the plurality of objective attributes comprises the vaginal fluid level value.

19. The system of claim 18, wherein measuring the vaginal fluid level value comprises optical coherence angiography, Doppler OCT, or a lymphatic vessel visualization technique.

20. The system of claim 12, wherein the memory component further comprises instructions for:
a. measuring, based on an optical sensing technique, an electrical sensing technique, or a combination thereof, a pH measurement;
wherein the plurality of objective attributes comprises the pH measurement.
